(19) United States
(12) Patent Application Publication
(10) Pub. No.: US 2023/0053337 A1
(43) Pub. Date: Feb. 23, 2023

(54) DELIVERY GUIDEWIRE AND THERAPEUTIC TREATMENT DEVICE

(71) Applicant: MICROPORT NEUROTECH (SHANGHAI) CO., LTD., Shanghai (CN)

(72) Inventors: Ping LONG, Shanghai (CN); Juan HOU, Shanghai (CN); Hao TIAN, Shanghai (CN)

(21) Appl. No.: 17/409,085

(22) Filed: Aug. 23, 2021

(51) Int. Cl. A61M 25/09; A61M 25/00; A61L 29/18; A61L 29/04; A61L 29/02; A61F 2/962

(52) U.S. Cl. CPC A61M 25/09; A61F 2/962; A61L 29/02; A61L 29/18; A61L 29/041; A61M 25/0012; A61M 2025/09083; A61M 2025/09133; A61M 2025/09166

(57) ABSTRACT

The present invention relates to a delivery guidewire and a therapeutic treatment device including the delivery guidewire. The delivery guidewire includes a core shaft and a driving member arranged on the core shaft, and the driving member includes inner and outer components. The inner component is made of a metal and fixedly sleeved over the core shaft, and the outer component is made of a polymeric material and fixedly sleeved over the inner component. The inner component fixedly sleeved over the core shaft indirectly enhances attachment of the outer component to the core shaft, thus reducing the risk of loosening, wrinkling or displacement of the outer component and resulting in improved safety and reliability of the delivery guidewire during use.

### TECHNICAL FIELD

The present invention relates to the technical field of medical instruments and, more specifically, to a delivery guidewire and a therapeutic treatment device.

### BACKGROUND

Most intracranial aneurysms are visualized as abnormal encephalocele in the walls of cerebral arteries and are the No. 1 cause of subarachnoid hemorrhage. Among cerebrovascular diseases, with incidence being second only to that of cerebral thrombosis and hypertensive cerebral hemorrhage, intracranial aneurysms are extremely risky and dangerous.

Currently, there are essentially three options for treating intracranial aneurysms: 1) surgical clipping, involving blocking cerebral blood circulation to the aneurysm by clipping the base thereof with a metal clip, thus not only restoring the parent artery’s normal blood supply of the parent artery but also preventing the aneurysm from rupture and consequential bleeding; 2) intra-aneurysmal embolization, involving placing an embolism material in the aneurysm for embolization thereof, which can prevent further expansion of the aneurysm that may ultimately lead to its rupture and consequential bleeding; and 3) endovascular stenting, involving implanting a stent into the artery to reduce blood flow therein to the aneurysm, thus causing blood stagnation and thrombus formation in the aneurysm, which facilitates the closure of the aneurysm and lowers the risk of rupture. As aneurysms typically occur around the circle of Willis where there are many important blood vessels, nerves and brain tissues, the surgical clipping of an aneurysm is very challenging to the operating physician, and the patient mortality rate has been found to reach as high as 50%. For complex aneurysms, such as large and giant ones, simple reliance on intra-aneurysmal embolization is problematic because frequent recurrence has been confirmed. For these reasons, endovascular stenting is most commonly chosen for the treatment of intracranial aneurysms.

Endovascular stenting of an intracranial aneurysm involves delivering a stent into blood vessels by using a delivery guidewire including a core shaft and a membrane structure arranged on the core shaft. The stent is loaded on the membrane structure so that it can move in sync with the delivery guidewire by virtue of friction with the membrane structure. Conventionally, the membrane structure is directly attached to the core shaft by adhesive bonding. However, this approach suffers from weak attachment due to a relatively small contact area between the membrane structure and the core shaft, which tends to lead to loosening, wrinkling or displacement of the membrane structure, or even dislodgement of the stent, during delivery thereof.

### SUMMARY OF THE DISCLOSURE

It is an objective of the present invention to provide a delivery guidewire and a therapeutic treatment device. The delivery guidewire has good flexibility that allows effective avoidance of dislodgement of a stent being delivered due to loosening or displacement of an outer component.

To this end, the present invention provides a delivery guidewire, comprising a core shaft and a driving member arranged on the core shaft, the driving member comprising an inner component and an outer component,
wherein the inner component is made of a metal and fixedly sleeved over the core shaft, and
wherein the outer component is made of a polymeric material and fixedly sleeved over the inner component.

Optionally, the inner component has a recess that is totally or partially filled by the outer component.

Optionally, the outer component at least partially extends in the recess to connect with the core shaft.

Optionally, a groove is defined in an outer surface of the inner component to form the recess.

Optionally, the inner component comprises a plurality of coils arranged on the core shaft along an axis thereof, and the recess is formed between any adjacent two of the coils.

Optionally, the inner component has a spiral structure formed by spirally winding a wire on the core shaft along an axis thereof, and the recess is formed between adjacent turns of the spiraled wire.

Optionally, the inner component has a meshed tubular structure braided from wires, and the recess is formed by an opening of the meshed tubular structure.

Optionally, each of the wires has a diameter of 0.001 inch or less.

Optionally, the meshed tubular structure has a plurality of intersections formed by the wires, and a number of the intersections ranges from 15 to 50 per inch of the meshed tubular structure.

Optionally, the inner component comprises at least one tubular element that is entirely or partially wrapped by the outer component.

Optionally, the tubular element has a recess that is partially or entirely filled by the outer component, or

wherein the inner component comprises at least two tubular elements, a recess is formed between any adjacent two of the tubular elements, and the recess is partially or entirely filled by the outer component.

Optionally, the inner component is formed of a radiographically visible metal.

Optionally, the metal is one or more selected from a group comprising platinum, gold, tungsten, a platinum-gold alloy, a platinum-tungsten alloy, a platinum-iridium alloy and a platinum-nickel alloy.

Optionally, the inner component is welded or adhesively bonded to the core shaft.

Optionally, the outer component is made of a material selected from any one or more of a group comprising a block polyether amide resin, a thermoplastic polyurethane elastomer, silicone, nylon and an acrylic polymer.

Optionally, the outer component wraps the inner component and extends to connect with the core shaft.

Optionally, the outer component is formed on the inner component by hot pressing and/or dip-coating, or adhesively attached to the inner component.

Optionally, the inner component is integrally formed with the core shaft.

Optionally, at least two said driving members are provided on the core shaft, and the at least two driving members are spaced from one another along an axis of the core shaft.

To achieve the above objective, the present invention also provides a therapeutic treatment device, comprising a delivery catheter, a medical implant and the above delivery guidewire, wherein the delivery catheter has an inner cavity extending therethrough axially, and the inner cavity is configured to receive the medical implant therein, wherein the medical implant is sleeved on the driving member and compressed in the inner cavity against a wall thereof.

Optionally, the inner cavity has a radial dimension ranging from 0.017 inches to 0.029 inches.

Compared with the prior art, the delivery guidewire and therapeutic treatment device of the present invention offer the following advantages:
The delivery guidewire includes a core shaft and a driving member arranged on the core shaft. The driving member includes an inner component made of metal and an outer component made of polymeric material. The inner component is fixedly sleeved over the core shaft, and the outer component is fixedly sleeved over the inner component. Since the inner component is fixed (e.g., welded) to the core shaft, and both of them are made of metal, they can be attached together very strongly without any potential relative displacement between them. In addition, the fixed attachment of the polymeric outer component to the inner component is accomplished by a special structural design. For example, a recess may be defined in the inner component, and entirely or partially filled by the outer component. In this way, an intermeshing fit can be achieved between the inner and outer components. As another example, the outer component may wrap the inner component and extend to be coming into attachment to the core shaft so that the outer component, the inner component and the core shaft are tightly attached together. Arranging the outer component over the core shaft through the inner component that has a relatively large contact area with the outer component allows increased attachment strength. Compared with the conventional ones, the design according to the present invention enables firm attachment of the whole driving member to the core shaft, thus avoiding loosening, wrinkling or displacement of the driving member during delivery of the stent. As a result, improved reliability of the delivery guidewire during delivery of the medical implant is achieved.

### BRIEF DESCRIPTION OF THE DRAWINGS

FIG. 1 is a diagram schematically illustrating the structure of a conventional therapeutic treatment device.

FIG. 2 is a diagram schematically illustrating the structure of a delivery guidewire according to an embodiment of the present invention.

FIG. 3 is a partial cross-sectional view of a delivery guidewire according to an embodiment of the present invention, showing multiple coils arranged adjacent to one another.

FIG. 4 is a schematic enlarged view of portion A of the delivery guidewire of FIG. 3.

FIG. 5 schematically illustrates the structure of a delivery guidewire according to an embodiment of the present invention, showing only part of a core shaft and an inner component disposed over the core shaft.

FIG. 6 is a structural schematic of a delivery guidewire according to another embodiment of the present invention.

FIG. 7 is a schematic enlarged view of portion B of the delivery guidewire of FIG. 6.

FIG. 8 is a structural schematic of a delivery guidewire according to yet another embodiment of the present invention.

FIG. 9 is a schematic enlarged view of portion C of the delivery guidewire of FIG. 8.

FIG. 10 is a structural schematic of a delivery guidewire according to a further embodiment of the present invention.

### LIST OF REFERENCE NUMBERS IN DRAWINGS

10, 100 Delivery Guidewire
11, 110 Core Shaft
12 Membrane Structure
120 Driving Member
121 Inner Component
122 Outer Component
123 Recess
130 First Radiopaque Member
140 Second Radiopaque Member
150 Diametrically-Varying Spring
20 Stent
30 Delivery Catheter

### DETAILED DESCRIPTION

Objects, advantages and features of the present invention will become more apparent from the following more detailed description thereof made in conjunction with the accompanying drawings. Note that the figures are provided in a very simplified form not necessarily drawn to exact scale for the only purpose of helping to explain the disclosed embodiments in a more convenient and clearer way.

As used herein, the singular forms “a”, “an” and “the” include plural referents, and the term “multiple” means two or more, unless the context clearly dictates otherwise. As used herein, the term “or” is employed in the sense including “and/or” unless the context clearly dictates otherwise. Moreover, the terms “installation”, “connection” and “coupling” should be interpreted in a broad sense. For example, a connection may be a permanent connection, a detachable connection, or an integral connection. It may also be a mechanical connection or an electrical connection. Further, it may be also be a direct connection, an indirect connection with one or more intervening elements, or an internal communication or interaction between two components. Those of ordinary skill in the art would readily understand these terms based on the context in which they are used. Throughout the figures, like elements are given the same or analogous reference numbers.

As used herein, a “proximal end” and a “distal end” are relative orientations, relative positions and directions of elements or actions relative to each other from the perspective of a surgeon using the medical instruments. Although the “proximal end” and the “distal end” are not restrictive, the “proximal end” generally refers to the end of the medical device that is close to the surgeon during normal operation, while the “distal end” generally refers to the end that first enters the patient’s body.

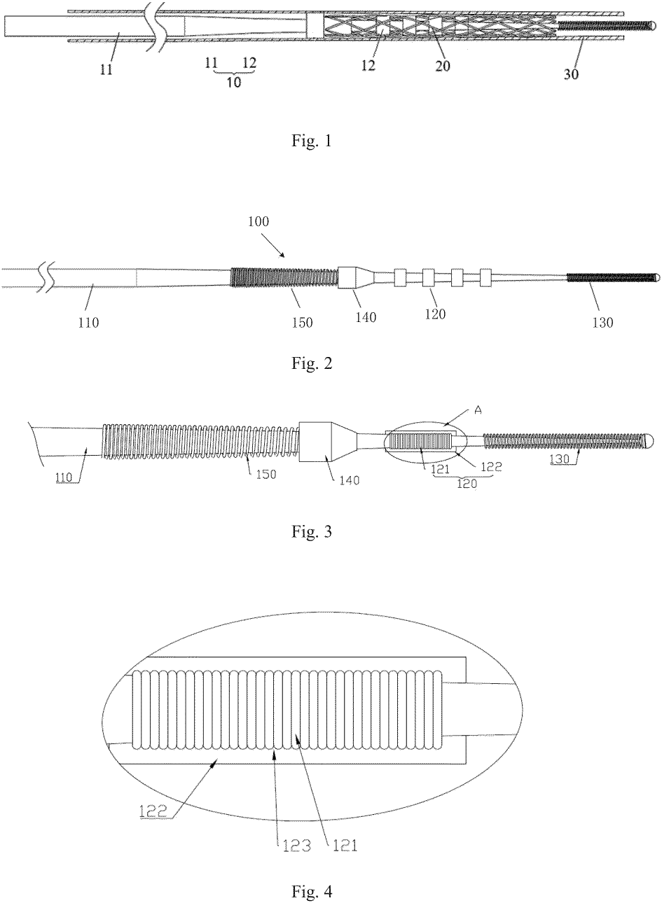

FIG. 1 is a diagram schematically illustrating the structure of a conventional therapeutic treatment device for delivering a medical stent to a target site in the body of a patient. As shown in FIG. 1, the therapeutic treatment device includes a delivery guidewire 10, a stent 20 and a delivery catheter 30. The delivery guidewire 10 includes a core shaft 11 and a membrane structure 12 arranged on the core shaft 11. The delivery catheter 30 defines an inner cavity, which extends therethrough axially and is configured to receive the stent 20. The stent 20 is compressed against the wall of the inner cavity and is thus sleeved over the membrane structure 12. In order to be smoothly moved within the delivery catheter 30, this conventional delivery guidewire 10 has a smooth outer surface, while the membrane structure 12 is arranged on the core shaft 11 of the delivery guidewire 10 to enhance a first friction force between the delivery guidewire 10 and the stent 20. Generally, the membrane structure 12 is made of a polymeric material with a relatively high coefficient of friction and is adhesively bonded to the core shaft 11 that is fabricated from a metal.

In this therapeutic treatment device, the delivery catheter 30, the stent 20 and the delivery guidewire 10 are assembled together by means of interference fits. In other words, the delivery catheter 10 applies a radial force onto the stent 20, and the stent 20 applies a radial force onto the delivery guidewire 10 (more exactly, the membrane structure 12). When the operator pushes the delivery guidewire 10 to cause an axial movement thereof in the delivery catheter 30, a first friction force created between the delivery guidewire 10 and an inner surface of the stent 20 drives the stent 20 to move in sync with the delivery guidewire 10. At the same time, a second friction force is created between an outer surface of the stent 20 and an inner wall of the delivery catheter 30. However, attachment between the membrane structure 12 and the core shaft 11 tends to be weak due to a relatively small diameter of the core shaft 11 that leads to a limited adhesive attachment area between the membrane structure 12 and the core shaft 11 and due to the fact that the core shaft 11 and the membrane structure 12 are made of different materials. Consequently, when the second friction force goes high, it is very easy for the membrane structure 12 to loosen, wrinkle, displace, or even cause dislodgement of the stent 20.

In order to overcome the above problems, it is an objective of embodiments of the present invention to provide a delivery guidewire for delivering a medical implant to a target site in a patient’s body with a significantly reduced risk of dislodgement of the medical implant during delivery and increased safety and reliability. The medical implant is for example, a self-expanding (or self-expandable) stent such as a braided stent or a cut stent. In alternative embodiments, the medical implant may be a medical embolization coil, blood vessel occluder or the like. However, the present invention is not limited to any particular type of the medical implant. In the following, for ease of explanation, the medical implant is described as a self-expanding stent by way of example, which is referred to as the “stent” hereinafter for the sake of brevity.

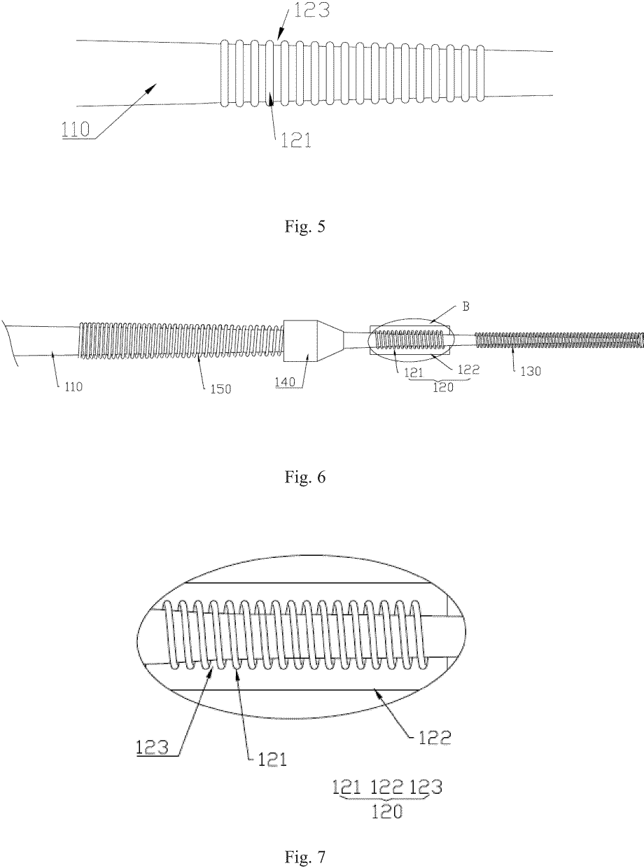

Referring to FIGS. 2 to 4, a delivery guidewire 100 according to an embodiment of the present invention includes a core shaft 110 and a driving member 120 arranged on the core shaft 110. The driving member 120 includes an inner component 121 and an outer component 122. The inner component 121 is made of a metal and fixedly sleeved over the core shaft 110, while the outer component 122 is made of a polymeric material and fixedly sleeved over the inner component 121.

The metal inner component 121 may be adhesively bonded, welded or otherwise attached to the core shaft 110 with attachment strength that is high enough to disallow displacement of the inner component 121 on the core shaft 110, and the arrangement of the outer component 122 attached to the core shaft 110 by the inner component 121 indirectly allows the outer component 122 to have an extended attachment area with the core shaft 110, which results in enhanced attachment of the outer component 122 to the core shaft 110 and a reduced risk of loosening, wrinkling or displacement of the outer component 122 during delivery of the stent. It would be appreciated that, as used herein, the phrase “sleeved over” means either that the inner component 121 is separate from the core shaft 110 and assembled with the same after they are fabricated, or that the two are integrally fabricated so that the inner component 121 extends outwardly from an outer surface of the core shaft 110.

Additionally, a recess 123 may be formed in the surface of the inner component 121 and totally or partially filled by the outer component 122, in order to achieve an extended attachment area between the outer component 122 and the inner component 121.

According to embodiments of the present invention, the inner component 121 may assume any of multiple forms, some preferred ones are explained below with reference to the annexed figures. It is to be understood that the various forms of the inner component 121 described below are only optional implementations of the present invention and should not be taken to limit the invention in any sense.

As shown in FIGS. 2 to 4, in one embodiment, the inner component 121 may consist of multiple coils disposed over the core shaft 110, which are arranged side by side along an axis of the core shaft 110. In this embodiment, the coils may be formed from a metal wire with a circular or elliptical cross section. Therefore, adjacent coils are brought into contact with each other, and quasi V-shaped groove, i.e., the recess 123, may be formed therebetween. In other embodiments, depending on the cross-sectional shape of the wire from which the coils are formed, the grooves may alternatively be U-shaped grooves, or cubic, rectangular parallelepiped or hemispherical pits.

The outer component 122 may be formed over an outer surface of the coil by hot pressing and/or dip-coating involving a process of molding, shaping and cooling. The process of the hot pressing may include disposing a polymer tube over the inner component 121 and a heat shrink tube over the polymer tube, heating the heat shrink tube and shaping it in a mold so that the melted material of the polymer tube flows into the recess 123 in the inner component 121, and then removing the heat shrink tube after the polymeric material is cooled down and cured. Examples of the material of the outer component 122 may include any of a thermoplastic elastomer such as a block polyether amide resin (PEBAX) or a thermoplastic polyurethane (TPU) elastomer, silicone, nylon, an acrylic polymer or another polymeric material, or combinations thereof. The polymeric material filled in the recess 123 allows a larger contact area and thus stronger attachment between the outer component 122 and the inner component 121. Before the polymeric material cures, it may even flow to the surface of the core shaft 110 between adjacent coils and/or beyond both ends of the inner component 121, resulting in more tight adhesive attachment between each two of the outer component 122, the inner component 121 and the core shaft 110 and an additional reduction in the risk of wrinkling, loosening or displacement of the outer component 122. In alternative embodiments, the outer component may be formed first, and then attached to the inner component 121 for example, by gluing.

Alternatively, as shown in FIG. 5, the multiple coils may be spaced over the core shaft 110, with the recess 123 being formed between adjacent coils. In this case, each two of the outer component 122, the inner component 121 and the core shaft 110 may be adhesively bonded together.

The inner component 121 may be wound on the core shaft 110 tightly enough to cause the inner component 121 to exert a large radial pressure on the core shaft 110 so that when the operator pushes the delivery guidewire 100, an increased friction force is created between the core shaft 110 and the inner component 121 and helps in maintaining the inner component 121 stationary with respect to the core shaft 110. The outer component 122 wraps the outer surface of the inner component 121, so that the outer component 122 is attached to the core shaft 110 via the inner component 121 with a large contact area between the outer component 122 and the inner component 121. The inner component 121 may be fixed to the core shaft 110 by welding (soldering or laser welding) with attachment strength much higher than that resulting from directly attaching a membrane structure to the core shaft 110. The wrapping of the outer component 122 on the inner component 121 may be accomplished by dip-coating or hot pressing. In an aspect, this brings the outer component 122 into contact with both the core shaft 110 and the inner component 121, resulting in increased attachment strength between them and reducing the risk of loosening, displacement or distortion of the outer component 122. In another aspect, the high attachment strength between the inner component 121 and the core shaft 110 can prevent the loosening or displacement of the driving member 120 as a whole with respect to the core shaft 110, thus effectively avoiding dislodgement of the stent. In alternative embodiments, the inner component 121 may also be fixed to the core shaft 110 by adhesive attachment (gluing) or any other suitable method.

Referring to FIGS. 6 and 7, in another embodiment of the present invention, the inner component 121 is a spiral structure formed by spirally winding a wire on the core shaft 110 along an axis thereof. Similar to the previous embodiment, adjacent turns of this spiral inner component 121 may be brought into contact with, or spaced apart from, each other.

Optionally, the wire from which the inner component 121 of this embodiment is formed may be a polymeric or metal wire. In the latter case, the resulting spiral structure may be flexible and easily bendable, making the delivery guidewire 100 desirably compliant as a whole.

In general terms, for a longer stent (i.e., a stent with a larger axial dimension) to be delivered, the driving member 120 on the core shaft 110 may be designed with a greater length, in order for a sufficient friction force to be created between the stent and the delivery guidewire 100. In this embodiment, a plurality of the driving members 120 may be arranged, in particular spaced, over the core shaft 110 along the axis thereof to obtain a larger total length of the driving members 120.

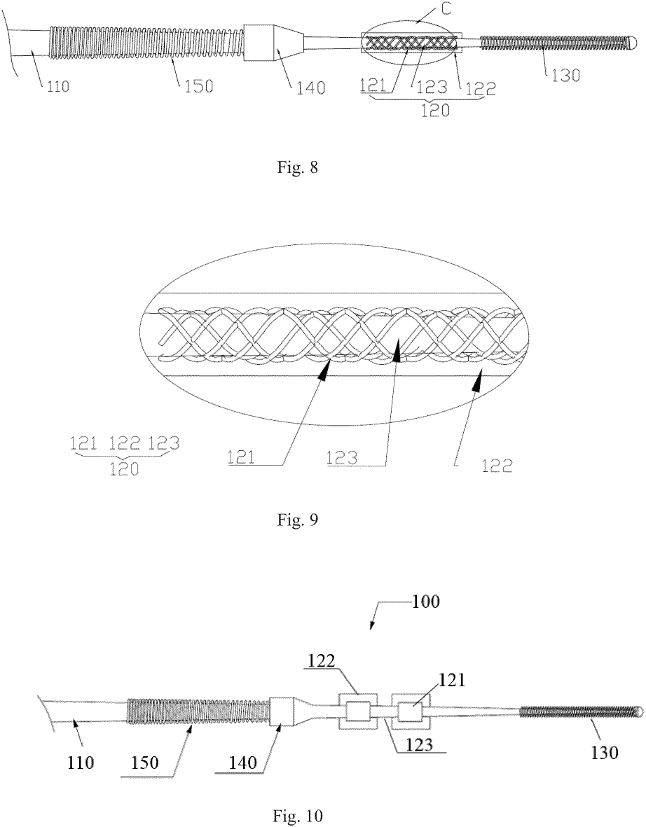

Referring to FIGS. 8 and 9, in a further embodiment of the present invention, the inner component 121 is a meshed tubular structure braided from wires. In this case, openings in the meshed tubular structure serve as the recess 123.

In this embodiment, one or more such driving members 120 may be provided on the core shaft 110. Each driving member 120 may be braided from wires that are as thin as applicable. For example, the wires may have a diameter (or cross-sectional width) of 0.001 inch or less. Additionally, the wires may be so braided that there is only a small number, preferably 15-50, intersections per inch of the inner component 121. In this way, the driving member(s) 120 may be firmly attached to the core shaft 110 while not compromising its flexibility.

FIG. 10 is a schematic illustration of a further embodiment of the present invention. As shown in FIG. 10, one or more inner components 121, each implemented as a metal tube, may be welded onto the core shaft 110. In this embodiment, each metal tube may be a tubular member with a neat surface. A length of each metal tube may be 0.3-2 mm in order to not adversely affect the flexibility of the delivery guidewire 100. When two such metal tubes are provided, the outer component 122 may be designed to entirely wrap both the metal tubes, as well as a bare surface portion of the core shaft 110 between the metal tubes (which serves as the recess 123), while further extending over the core shaft 110. Alternatively, each metal tube, i.e., each inner component 121, may be wrapped by a separate outer component 122 (see FIG. 10). In other words, the individual inner components 121 are wrapped with respective outer components 122. In addition, each of the outer components 122 may wrap part of the core shaft 110 between the metal tubes (which serves as the recess 123). The inner components 121 may be wrapped by the outer components 122 through dip-coating or hot pressing.

In alternative embodiments, each metal tube may have pits, slots or through holes formed, for example, by laser etching. This can further enlarge the recess 123 and result in an even larger contact area and enhanced attachment strength between the outer component 122 and the inner component 121.

In the preceding embodiment, the inner component 121 may be formed of a radiographically invisible or visible metal. Examples of the radiographically invisible metal may include, but are not limited to, stainless steel. Examples of the radiographically visible metal may include, but are not limited to, a platinum-tungsten or platinum-iridium alloy. Preferably, the inner component 121 is formed of a radiographically visible (or radiopaque) metal so that the driving member 120 is visible in a radiographic manner. More specifically, the inner component may be formed of one or more selected from the group consisting of platinum, gold, tungsten, a platinum-gold alloy, a platinum-tungsten alloy, a platinum-iridium alloy and a platinum-nickel alloy. For example, it may be formed of either or both of a platinum-tungsten alloy and a platinum-iridium alloy (in the latter case, for example, it may be a meshed tubular structure braided both from wires of the platinum-tungsten alloy and from wires of the platinum-iridium alloy). Advantageously, this allows the operator to accurately determine whether the stent being delivered is retrievable or not. In particular, in practice, the delivery catheter in which the stent is compressed on the delivery guidewire may have a first proximal end and a first distal end opposing the first proximal end, and a radiopaque ring (not shown) may be provided at the first distal end. During delivery, upon the driving member 120 coming into coincidence with the radiopaque ring, as viewed in a radiographic image, the operator may know that the stent cannot be retrieved anymore if it is further pushed forward distally. Therefore, the radiographic visibility of the driving member 120 allows accurate location with the aid of the radiographic imaging device, which greatly facilitates the operator’s operation.

With continued reference to FIG. 2, similar to the existing delivery guidewires, the delivery guidewire 100 of the present invention may further include a first radiopaque member 130 and a second radiopaque member 140. The core shaft 110 may have a second proximal end and a second distal end opposing the second distal end. The first radiopaque member 130 may be implemented as a radiopaque spring disposed at the second distal end, while the second radiopaque member 140 may be disposed on the core shaft 110. The driving member 120 may be disposed between the first radiopaque member 130 and the second radiopaque member 140. That is, in the therapeutic treatment device, the stent may be loaded between the first radiopaque member 130 and the second radiopaque member 140. During delivery of the stent, the operator may determine the location of the stent from those of the first radiopaque member 130 and the second radiopaque member 140.

The core shaft 110 may include at least one diametrically-varying section (not labeled) and at least one diametrically-constant section (also not labeled). In the direction from the second proximal end to the second distal end, these diametrically-constant section and diametrically-varying section may be alternately arranged and connected together. Each diametrically-varying section may have a third proximal end and a third distal end opposing the third proximal end and may be tapered inwardly from the third proximal end to the third distal end. Each diametrically-varying section may be connected at the third proximal end to a diametrically-constant section whose diameter is greater than that of a diametrically-constant section connected to the third distal end of the same diametrically-varying section. Due to the diametrically-varying section(s), the delivery guidewire 100 generally appears as a tapered structure that imparts, to the delivery guidewire, increased flexibility, force transmissibility and trackability, which are favorable to the delivery and deploy of the stent.

The second radiopaque member 140 may have a maximum outer diameter that is approximately equal to, or slightly smaller than, an inner diameter of the delivery catheter. The second radiopaque member 140 may have a fourth proximal end and a fourth distal end opposing the fourth proximal end. Conventionally, an outer diameter of a portion of the core shaft 110 close to the fourth proximal end of the second radiopaque member 140 is designed to be smaller than an inner diameter of the delivery catheter, leaving a clearance between the core shaft 110 and the delivery catheter. If this clearance is rather large, it may make the second distal end of the core shaft 110 lose stability when the delivery guidewire 100 is being pushed forward. This may increase the resistance to the advancement of the delivery guidewire 100. To overcome this, the core shaft 110 is modified to have a diametrically-varying section adjacent to the fourth proximal end of the second radiopaque member 140 and a diametrically-varying spring 150 disposed over the diametrically-varying section. Additionally, the diametrically-varying spring 150 is attached to the second radiopaque member 140. The diametrically-varying spring 150 is filled in the clearance between the core shaft 110 and the delivery catheter to ensure good stability of the second distal end of the core shaft 110 during delivery while not exerting any adverse effect on the flexibility of the core shaft 110. Optionally, the second radiopaque member 140 may include a main body defining a receptacle bore (not labeled) extending along an axis thereof. The receptacle bore may be tapered inwardly along the direction from the fourth proximal end to the fourth distal end, and there may be a clearance between the core shaft 110 and the wall of the receptacle bore at a portion thereof close to the first proximal end. A distal end portion of the diametrically-varying spring 150 may be received in the second radiopaque member 140 so that the diametrically-varying spring 150 is coaxial with the core shaft 110. Further, in embodiments of the present invention, in order to ensure sufficient flexibility of the core shaft 110 at the second distal end while not adversely affecting the transmission of any driving force, in the direction from the second proximal end to the second distal end, each turn-to-turn spacing may be greater than any more distal one and smaller than any more proximal one (i.e., the turns of the diametrically-varying spring 150 may become increasingly sparse in the direction from the second proximal end to the second distal end).

It is another objective of the present invention to provide a therapeutic treatment device including a delivery catheter, a medical implant and the delivery guidewire according to any of the above embodiments. The medical implant is disposed over the driving member, and the delivery catheter has an inner cavity extending therethrough axially. The inner cavity is configured to receive the delivery guidewire therein in such a manner that the medical implant is compressed in the inner cavity against a wall thereof. In embodiments of the present invention, the medical implant may be implemented as a self-expanding stent such as a braided or cut stent. Alternatively, the medical implant may be a spring coil, a blood vessel occluder, etc.

A recess 123 may be formed in a surface of the inner component 121, and the outer component 122 is at least partially filled in the recess 123. This allows enlarged contact areas, and thus increased friction forces creatable, between the outer component 122 and the core shaft 110 and the inner component 121 at a given contact area between the outer component 122 and the medical implant. In other words, filling at least part of the outer component 122 in the recess 123 allows a reduced outer diameter of the outer component 122, making it applicable to delivery catheters with various inner diameters. In this embodiment, there may be many options for the size of the inner cavity. For example, a radial dimension of the inner cavity may be in the range from 0.017 inches to 0.029 inches. Alternatively, the radial dimension of the inner cavity may be smaller, for example, 0.027 inches or less, or even 0.021 inches or less.

According to embodiments of the present invention, the driving member 120 is designed to include an inner component 121 made of a metal and an outer component 122 made of polymeric material, and the inner component 121 is fixedly sleeved over the core shaft 110, and the outer component 122 is fixedly sleeved over the inner component 121. In this design, the inner component 121 indirectly results in enhanced attachment between the outer component 122 and the core shaft 110, which reduces the risk of loosening, wrinkling or displacement of the driving member 120 during delivery of the stent. As a result, improved safety and reliability of the delivery guidewire 100 during the delivery of the medical implant is achieved.

Although the present invention has been disclosed as above, it is not limited to the above disclosure in any sense. Various changes and modifications can be made by those skilled in the art to the present invention without departing from the spirit and scope thereof. Accordingly, it is intended that any and all such changes and modifications are also embraced within the scope of the invention as defined in the appended claims and equivalents thereof.

What is claimed is:

1. A delivery guidewire, comprising a core shaft and a driving member arranged on the core shaft, the driving member comprising an inner component and an outer component,
wherein the inner component is made of a metal and fixedly sleeved over the core shaft, and
wherein the outer component is made of a polymeric material and fixedly sleeved over the inner component.

2. The delivery guidewire of claim 1, wherein the inner component has a recess that is totally or partially filled by the outer component.

3. The delivery guidewire of claim 2, wherein the outer component at least partially extends in the recess to connect with the core shaft.

4. The delivery guidewire of claim 2, wherein a groove is defined in an outer surface of the inner component to form the recess.

5. The delivery guidewire of claim 2, wherein the inner component comprises a plurality of coils arranged on the core shaft along an axis thereof, and the recess is formed between any adjacent two of the coils.

6. The delivery guidewire of claim 2, wherein the inner component has a spiral structure formed by spirally winding a wire on the core shaft along an axis thereof, and the recess is formed between adjacent turns of the spiraled wire.

7. The delivery guidewire of claim 2, wherein the inner component has a meshed tubular structure braided from wires, and the recess is formed by an opening of the meshed tubular structure.

8. The delivery guidewire of claim 7, wherein each of the wires has a diameter of 0.001 inch or less; the meshed tubular structure has a plurality of intersections formed by the wires, and a number of the intersections ranges from 15 to 50 per inch of the meshed tubular structure.

9. The delivery guidewire of claim 1, wherein the inner component comprises at least one tubular element that is entirely or partially wrapped by the outer component.

10. The delivery guidewire of claim 9, wherein the tubular element has a recess that is partially or entirely filled by the outer component, or
wherein the inner component comprises at least two tubular elements, a recess is formed between any adjacent two of the tubular elements, and the recess is partially or entirely filled by the outer component.

11. The delivery guidewire of claim 1, wherein the inner component is formed of a radiographically visible metal.

12. The delivery guidewire of claim 11, wherein the metal is one or more selected from a group comprising platinum, gold, tungsten, a platinum-gold alloy, a platinum-tungsten alloy, a platinum-iridium alloy and a platinum-nickel alloy.

13. The delivery guidewire of claim 1, wherein the inner component is welded or adhesively bonded to the core shaft.

14. The delivery guidewire of claim 1, wherein the outer component is made of a material selected from any one or more of a group comprising a block polyether amide resin, a thermoplastic polyurethane elastomer, silicone, nylon and an acrylic polymer.

15. The delivery guidewire of claim 1, wherein the outer component wraps the inner component and extends to connect with the core shaft.

16. The delivery guidewire of claim 1, wherein the outer component is formed on the inner component by hot pressing and/or dip-coating, or adhesively attached to the inner component.

17. The delivery guidewire of claim 1, wherein the inner component is integrally formed with the core shaft.

18. The delivery guidewire of claims 1, wherein at least two said driving members are provided on the core shaft, and the at least two driving members are spaced from one another along an axis of the core shaft.

19. A therapeutic treatment device, comprising a delivery catheter, a medical implant and the delivery guidewire of claim 1, wherein the delivery catheter has an inner cavity extending therethrough axially, and the inner cavity is configured to receive the medical implant therein, wherein the medical implant is sleeved on the driving member and compressed in the inner cavity against a wall thereof.

20. The therapeutic treatment device of claim 19, wherein the inner cavity has a radial dimension ranging from 0.017 inches to 0.029 inches.
